(19) United States
(12) Patent Application Publication
(10) Pub. No.: US 2023/0277182 A1
(43) Pub. Date: Sep. 7, 2023

(54) OCCLUSIVE IMPLANT SYSTEM

(71) Applicant: BOSTON SCIENTIFIC SCIMED, INC., Maple Grove, MN (US)

(72) Inventors: Jan-Hung Chen, St. Paul, MN (US); David John Onushko, Maple Grove, MN (US); Nicholas Lee Tassoni, Andover, MN (US); Christopher J. Clark, St. Michael, MN (US); Harishankar Natesan, Shoreview, MN (US)

(73) Assignee: BOSTON SCIENTIFIC SCIMED, INC., Maple Grove, MN (US)

(21) Appl. No.: 18/116,406

(22) Filed: Mar. 2, 2023

Related U.S. Application Data
(60) Provisional application No. 63/316,208, filed on Mar. 3, 2022.

(51) Int. Cl. A61B 17/12

(52) U.S. Cl. CPC A61B 17/12022; A61B 2017/1205

(57) ABSTRACT

An occlusive implant system may include an access device including a handle and an access sheath extending distally therefrom. The access device includes a working lumen extending longitudinally therethrough. A proximal portion of the working lumen defines a garage section and a distal portion of the working lumen extends distally of the garage section. The occlusive implant system may include an occlusive implant device including a delivery sheath configured to be received within the garage section, the delivery sheath having a delivery lumen, a core wire disposed therein, and an occlusive implant releasably attached to the core wire. The garage section has a first inner diameter and the distal portion of the working lumen has a second inner diameter less than the first inner diameter. The delivery sheath has an outer diameter less than the first inner diameter and greater than the second inner diameter.

### Cross-Reference to Related Applications

This application claims the benefit of priority of U.S. Provisional Application No. 63/316,208 filed Mar. 3, 2022, the entire disclosure of which is hereby incorporated by reference.

### TECHNICAL FIELD

The disclosure relates generally to medical devices and more particularly to medical devices that are adapted for use in percutaneous medical procedures including implantation into the left atrial appendage (LAA) of a heart.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

In one example, an occlusive implant system may comprise an access device including a handle and an access sheath extending distally from the handle, wherein the access device includes a working lumen extending longitudinally through the access sheath and the handle. A proximal portion of the working lumen defines a garage section connected to a proximal port of the handle, and a distal portion of the working lumen extends distally of the garage section to a distal end of the access sheath. The occlusive implant system may comprise an occlusive implant device including a delivery system including a delivery sheath configured to be slidably received within the garage section, the delivery sheath having a delivery lumen extending proximally from a distal end of the delivery sheath, a core wire slidably disposed within the delivery lumen, and an occlusive implant releasably attached to a distal end of the core wire, the occlusive implant being configured to shift between a delivery configuration and a deployed configuration. The occlusive implant is disposed within a distal portion of the delivery lumen in the delivery configuration. The garage section has a first inner diameter and the distal portion of the working lumen has a second inner diameter less than the first inner diameter. The delivery sheath has an outer diameter less than the first inner diameter and greater than the second inner diameter.

In addition or alternatively to any example described herein, the distal portion of the delivery lumen has a third inner diameter and the third inner diameter is substantially equal to or less than the second inner diameter.

In addition or alternatively to any example described herein, the working lumen includes a tapered portion extending between the distal portion of the working lumen and the garage section.

In addition or alternatively to any example described herein, the tapered portion tapers from the first inner diameter proximate a proximal end of the tapered portion to the second inner diameter proximate a distal end of the tapered portion.

In addition or alternatively to any example described herein, the delivery system includes a proximal hub, a mid-hub, and a mid-shaft extending from the proximal hub to the mid-hub. The delivery sheath extends distally from the mid-hub.

In addition or alternatively to any example described herein, the delivery sheath has a first length from the mid-hub to the distal end of the delivery sheath. The garage section has a second length from the proximal port to a distal end of the garage section. The second length is less than 10% greater than the first length.

In addition or alternatively to any example described herein, the second length is less than 5% greater than the first length.

In addition or alternatively to any example described herein, the distal end of the delivery sheath is disposed proximate a distal end of the garage section when the delivery sheath is disposed within the garage section and the mid-hub is positioned adjacent the proximal port of the handle.

In addition or alternatively to any example described herein, the core wire has a length greater than a combined length of the delivery system and the access sheath when the distal end of the delivery sheath is disposed within the garage section.

In addition or alternatively to any example described herein, an occlusive implant system may comprise a steerable access device including a handle and an access sheath extending distally from the handle, wherein the steerable access device includes a working lumen extending longitudinally through the access sheath and the handle. A proximal portion of the working lumen defines a garage section connected to a proximal port of the handle, and a distal portion of the working lumen extends distally of the garage section to a distal end of the access sheath. The occlusive implant system may comprise an occlusive implant device including a delivery system including a delivery sheath configured to be slidably received within the garage section, the delivery sheath having a delivery lumen extending proximally from a distal end of the delivery sheath, a core wire slidably disposed within the delivery lumen, and an occlusive implant releasably attached to a distal end of the core wire, the occlusive implant being configured to shift between a delivery configuration and a deployed configuration. The occlusive implant is disposed within a distal portion of the delivery lumen in the delivery configuration. The garage section has a first inner diameter and the distal portion of the working lumen has a second inner diameter different from the first inner diameter.

In addition or alternatively to any example described herein, the working lumen changes from the first inner diameter to the second inner diameter within the handle.

In addition or alternatively to any example described herein, the garage section is disposed proximal of a distal end of the handle.

In addition or alternatively to any example described herein, the garage section is less than 10 inches in length from the proximal port to a distal end of the garage section.

In addition or alternatively to any example described herein, the distal end of the delivery sheath is prevented from extending distal of the garage section.

In addition or alternatively to any example described herein, a method of delivering an occlusive implant to a treatment site may comprise advancing a distal end of an access device to the treatment site, the access device including a handle, an access sheath extending distally from the handle, and a working lumen extending through the access sheath and the handle. A proximal portion of the working lumen defines a garage section connected to a proximal port of the handle and having a first inner diameter, and a distal portion of the working lumen extends distally of the garage section to a distal end of the access sheath and has a second inner diameter less than the first inner diameter. The method may comprise inserting a delivery sheath into the garage section and positioning a distal end of the delivery sheath proximate a distal end of the garage section, the delivery sheath having a delivery lumen extending proximally from the distal end of the delivery sheath. An occlusive implant is disposed within a distal portion of the delivery lumen in a delivery configuration, and a core wire releasably attached to the occlusive implant is slidably disposed within the delivery lumen. The method may comprise advancing the core wire distally through the delivery lumen and into the working lumen to advance the occlusive implant through the working lumen and out the distal end of the access device at the treatment site. The occlusive implant is configured to shift to a deployed configuration when unconstrained.

In addition or alternatively to any example described herein, the working lumen tapers from the first inner diameter to the second inner diameter within the handle.

In addition or alternatively to any example described herein, an outer diameter of the access sheath tapers radially inward in a distal direction within the handle.

In addition or alternatively to any example described herein, the distal end of the access sheath is steerable using a knob on the handle.

In addition or alternatively to any example described herein, the occlusive implant is a left atrial appendage closure device.

In addition or alternatively to any example described herein, the delivery sheath has an outer diameter greater than the second inner diameter and the delivery lumen has a third inner diameter substantially equal to or less than the second inner diameter.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description in connection with the accompanying drawings, in which:

FIGS. 1-2 are side views of selected aspects of a prior art occlusive implant device;

FIGS. 3-4 illustrate selected aspects of a prior art access device for delivering an occlusive implant to a treatment site;

FIGS. 5-6 illustrates selected aspects of a prior art occlusive implant system using the access device of FIGS. 3-4 and the occlusive implant device of FIGS. 1-2;

FIG. 7 illustrates selected aspects of an occlusive implant device according to the present disclosure;

FIG. 8 is a partial cross-sectional view illustrating selected aspects of an access device according to the present disclosure;

FIG. 8A is a partial cross-sectional view illustrating selected aspects of an alternative embodiment of an access device according to the present disclosure;

FIGS. 9-10 are partial cross-sectional views illustrating selected aspects of an occlusive implant system using the occlusive implant device of FIG. 7 and the access device of FIG. 8 according to the present disclosure;

FIG. 11 is a partial cross-sectional view illustrating selected aspects of the occlusive implant system of FIGS. 9-10;

FIG. 12 is a partial cross-sectional view illustrating selected aspects of an alternative configuration of the occlusive implant system of FIGS. 9-10; and

FIGS. 13-14 illustrate selected aspects of the occlusive implant device of FIG. 7.

While aspects of the disclosure are amenable to various modifications and alternative forms, examples are shown in the drawings and described herein. It should be understood, however, that the intention is not to limit aspects of the disclosure to the particular embodiments described. On the contrary, the disclosure shall cover all modifications, equivalents, and alternatives falling within the spirit and scope thereof.

### DETAILED DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following figures illustrate selected components and/or arrangements of an implant for occluding the left atrial appendage, a system for occluding the left atrial appendage, and/or methods of using the implant and/or the system. It should be noted that in any given figure, some features may not be shown, or may be shown schematically, for simplicity. Additional details regarding some of the components of the implant and/or the system may be illustrated in other figures in greater detail. While discussed in the context of occluding the left atrial appendage, the implant and/or the system may also be used for other interventions and/or percutaneous medical procedures within a patient. Similarly, the devices and methods described herein with respect to percutaneous deployment may be used in other types of surgical procedures, as appropriate. For example, in some examples, the devices may be used in a non-percutaneous procedure. Devices and methods in accordance with the disclosure may also be adapted and configured for other uses within the anatomy.

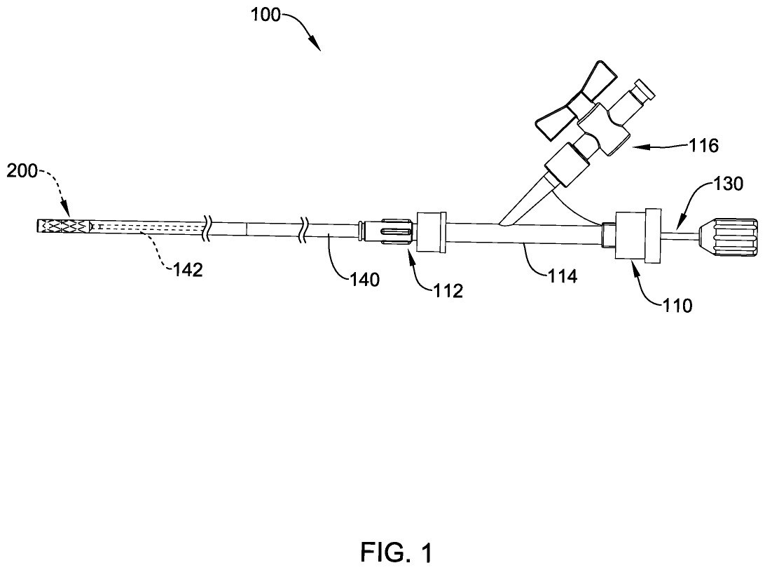

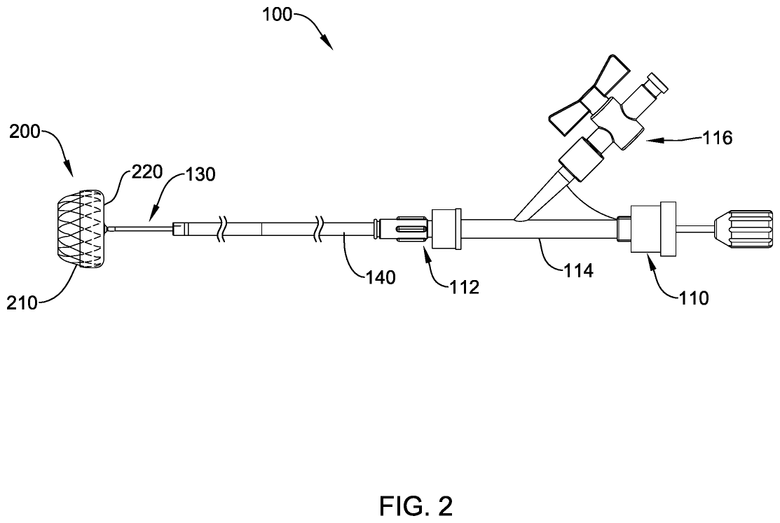

FIGS. 1-2 schematically illustrate selected components and/or arrangements of an occlusive implant device 100. It should be noted that in any given figure, some features of the occlusive implant device 100 may not be shown, or may be shown schematically, for simplicity. Additional details regarding some of the components of the occlusive implant device 100 may be illustrated in other figures in greater detail. The occlusive implant device 100 may be used to deliver and/or deploy a variety of medical implants (e.g., a cardiovascular implant, an occlusive implant, etc.) to one or more locations within the anatomy, including but not limited to, in some embodiments, the heart and/or the left atrial appendage. In the interest of clarity, the following discussion refers to an occlusive implant, but other medical implants may be used and/or considered with the occlusive implant device 100.

The occlusive implant device 100 may include a delivery system including a delivery sheath 140 having a delivery lumen 142 extending proximally from a distal end of the delivery sheath 140. In one example, the delivery lumen 142 extends from a proximal opening to a distal opening of the delivery sheath 140. The delivery system may include a proximal hub 110. The delivery system may include a mid-hub 112. The delivery system may include a mid-shaft 114 extending from the proximal hub 110 to the mid-hub 112. The delivery sheath 140 may extend distally from the mid-hub 112. The delivery system may include a side port 116. The delivery system and/or the delivery lumen 142 may include a proximal segment (not shown) extending within and/or through the mid-hub 112, the mid-shaft 114, and the proximal hub 110. The proximal segment may be in fluid communication with and/or may be an extension of the delivery lumen 142 of the delivery sheath 140. The side port 116 may be in fluid communication with the proximal segment and/or the delivery lumen 142.

The occlusive implant device 100 may include a core wire 130 slidably and/or rotatably disposed within the delivery lumen 142 and the proximal segment. The occlusive implant device 100 may include an occlusive implant 200, which may be configured for implantation within a left atrial appendage, releasably attached to a distal end of the core wire 130. In at least some embodiments, the occlusive implant 200 may be a left atrial appendage closure device. In some embodiments, a proximal end of the core wire 130 may extend proximally of a proximal end of the delivery sheath 140 and/or the proximal opening of the delivery lumen 142 for manual manipulation by a clinician or practitioner. Some suitable, but non-limiting, examples of materials for the occlusive implant device 100, the core wire 130, and/or the delivery sheath 140, etc., including but not limited to metallic materials, polymeric materials, etc., are discussed below.

The occlusive implant 200 may include an expandable framework 210 (e.g., FIG. 2) configured to shift between a delivery configuration (e.g., FIG. 1), such as when the occlusive implant 200 is disposed within the delivery lumen 142 proximate the distal opening and/or within a distal portion of the delivery lumen 142, to a deployed configuration (e.g., FIG. 2), when the occlusive implant 200 is unconstrained by the delivery sheath 140.

In some embodiments, the expandable framework 210 may comprise a plurality of interconnected struts. In some embodiments, the expandable framework 210 may be compliant and substantially conform to and/or be in sealing engagement with the shape and/or geometry of the left atrial appendage in the deployed configuration.

In some embodiments, a proximal end of the expandable framework 210 may be configured to releasably attach, join, couple, engage, or otherwise connect to the distal end of the core wire 130 (e.g., FIG. 2). In some embodiments, the proximal end of the expandable framework 210 may include a proximal hub coupled thereto. In some embodiments, the proximal hub may be configured to and/or adapted to releasably couple with, join to, mate with, or otherwise engage a distal end of the core wire 130. Other means of releasably coupling and/or engaging the proximal hub of the expandable framework 210 to the distal end of the core wire 130 are also contemplated.

In some embodiments, the occlusive implant 200 may include an occlusive element 220 (e.g., a membrane, a fabric, or a tissue element, etc.) connected to, disposed on, disposed over, disposed about, or covering at least a portion the expandable framework 210. In some embodiments, the occlusive element 220 may be connected to, disposed on, disposed over, disposed about, or cover at least a portion of an outer (or outwardly facing) surface of the expandable framework 210.

In some embodiments, the occlusive element 220 may be permeable or impermeable to blood and/or other fluids, such as water. In some embodiments, the occlusive element 220 may include a polymeric membrane, a metallic or polymeric mesh, a porous or semi-porous filter-like material, or other suitable construction. In some embodiments, the occlusive element 220 prevents thrombi (e.g., blood clots, etc.) from passing through the occlusive element 220 and out of the left atrial appendage into the blood stream. In some embodiments, the occlusive element 220 promotes endothelization after implantation, thereby effectively removing the target site (e.g., the left atrial appendage, etc.) from the patient's circulatory system. Some suitable, but non-limiting, examples of materials for the occlusive element 220 are discussed below.

In some embodiments, the expandable framework 210 and/or the plurality of interconnected struts may be integrally formed and/or cut from a unitary member. In some embodiments, the expandable framework 210 and/or the plurality of interconnected struts may be integrally formed and/or cut from a unitary tubular member and subsequently formed and/or heat set to a desired shape in the expanded configuration. In some embodiments, the expandable framework 210 and/or the plurality of interconnected struts may be integrally formed and/or cut from a unitary flat member or sheet, and then rolled or formed into a tubular structure and subsequently formed and/or heat set to the desired shape in the expanded configuration. Some exemplary means and/or methods of making and/or forming the expandable framework 210 include laser cutting, machining, punching, stamping, electro discharge machining (EDM), chemical dissolution, etc. Other means and/or methods are also contemplated.

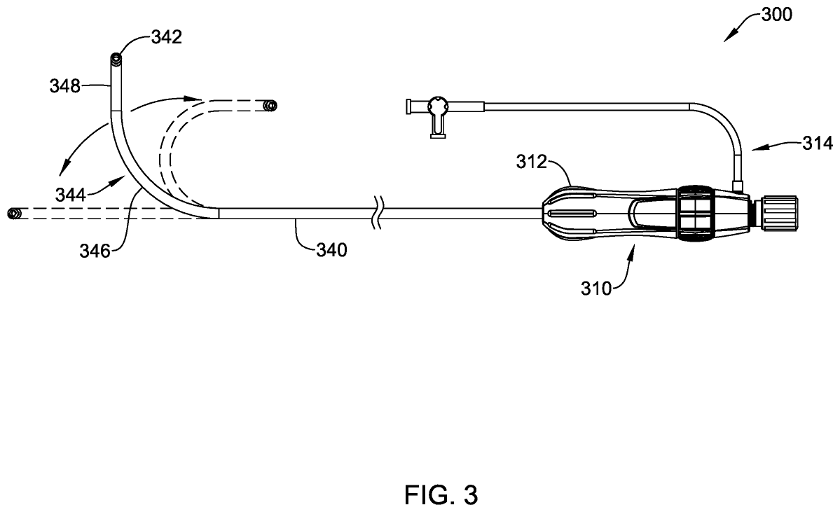

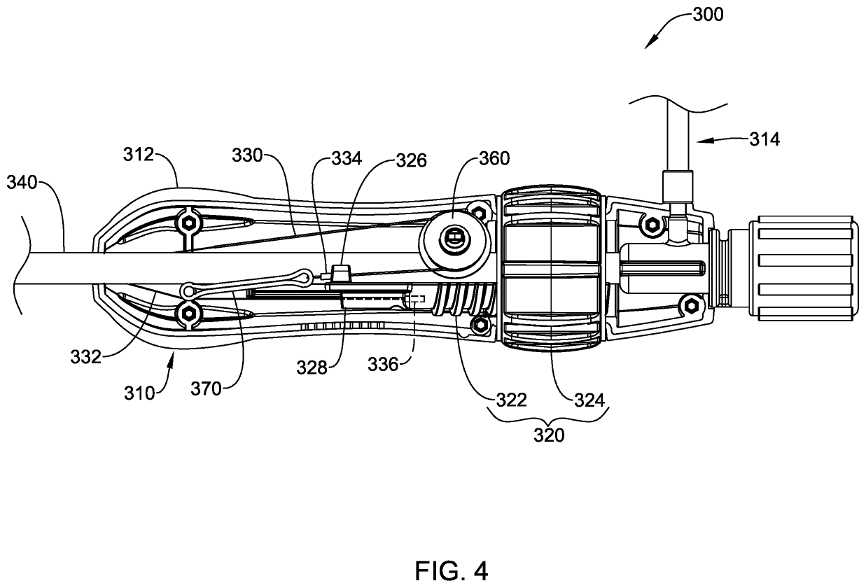

FIGS. 3-4 illustrate selected aspects of an access device 300. In one example, the access device 300 may be a bi-directional steerable catheter and/or an intravascular catheter. Examples of intravascular catheters may include, but are not limited to, balloon catheters, atherectomy catheters, device delivery catheters, drug delivery catheters, diagnostic catheters, and guide catheters.

The access device 300 may include a handle 310 and an access sheath 340 extending distally from the handle 310. The access sheath 340 may be a generally tubular elongate member. In some embodiments, the access device 300 and/or the handle 310 may include at least one port 314 connected thereto. In some embodiments, the at least one port 314 may include a guidewire port, a side port, a fluid flush port, an imaging access port, or other suitable ports, access points, or functional features. The handle 310 may include a handle housing 312. The access sheath 340 may extend into and/or through a distal opening in the handle housing 312. In at least some embodiments, a proximal end of the access sheath 340 may be fixedly attached to and/or inside of the handle housing 312. In some embodiments, the access sheath 340 may have a normal or relaxed configuration. The access sheath 340 may be self-biased toward, and/or in the absence of any outside forces may return to, the normal or relaxed configuration. Some suitable but non-limiting materials for the handle 310 and/or the handle housing 312 are described below.

In some embodiments, the access sheath 340 may include a soft and/or atraumatic distal tip 342. In some embodiments, the access sheath 340 may include a distal portion 344 having a first curve 346 and a second curve 348, such that the access sheath 340 has a preset double curve, in the normal or relaxed configuration. In some embodiments, the first curve 346 may be preset to curve upwards, as viewed from the side. Other configurations are also contemplated. In some embodiments, the second curve 348 may be preset to curve to the left, as viewed proximally to distally along the access sheath 340. Other configurations are also contemplated. In some embodiments, the distal portion 344 and/or the first curve 346 may be configured to bend or deflect in a first direction, wherein the distal tip 342 is bent and/or moved towards and/or closer to the handle 310, toward and/or to a deflected configuration, as shown in FIG. 3. In some embodiments, the distal portion 344 and/or the first curve 346 may be configured to bend or deflect in a second direction opposite the first direction, wherein the distal tip 342 is bent and/or moved away from and/or farther from the handle 310, toward and/or to a straightened configuration, as shown in FIG. 3. In some embodiments, the access sheath 340 may have only a single curve in the normal or relaxed configuration. In some embodiments, the access sheath 340 may be substantially straight in the normal or relaxed configuration.

In some embodiments, the access sheath 340 may be sized in accordance with its intended use. For example, the access sheath 340 can have a length that is in the range of about 25 to about 150 centimeters, about 50 to about 125 centimeters, or about 75 to about 100 centimeters. Other lengths are also contemplated. It is further contemplated that the outer diameter of the access sheath 340 may vary based on the use or application. In some examples, the outer diameter of the access sheath 340 may be about 2 millimeters (mm), about 3 mm (or 9 French), about 3.5 mm, about 4 mm (or 12 French), about 4.5 mm, about 5 mm (or 15 French), about 5.33 mm, about 5.5 mm, about 5.66 mm (or 17 French), about 6 mm, about 6.5 mm, about 7 mm (or 21 French), about 8 mm, or other suitable sizes. In some embodiments, the outer diameter of the access sheath 340 may be a maximum of 5.66 mm (17 French), and is preferably smaller than 5.66 mm (17 French). Other configurations are also contemplated. Some suitable but non-limiting materials for the access sheath 340 are described below.

In the view shown in FIG. 4, a portion of the handle housing 312 has been removed to show internal components of the handle 310. The handle 310 may include an axial translation mechanism 320. The axial translation mechanism 320 may include a threaded member 322 slidably disposed within the handle 310 and/or the handle housing 312. The axial translation mechanism 320 may include a rotatable knob 324. In some embodiments, the rotatable knob 324 may be disposed about and/or may be configured to rotate about, and/or relative to, at least a portion of the handle 310 and/or the handle housing 312. The rotatable knob 324 may be configured to engage the threaded member 322 such that rotation of the rotatable knob 324 relative to the handle 310 and/or the handle housing 312 causes axial translation of the threaded member 322 proximally and/or distally within the handle 310 and/or the handle housing 312. In some embodiments, rotation of the rotatable knob 324 in a clockwise direction, as viewed along the access device 300 proximally to distally, may cause axial translation of the threaded member 322 distally within the handle 310 and/or the handle housing 312. In some embodiments, rotation of the rotatable knob 324 in a counterclockwise direction, as viewed along the access device 300 proximally to distally, may cause axial translation of the threaded member 322 proximally within the handle 310 and/or the handle housing 312. In some embodiments, the reverse and/or opposite configuration may be used, wherein clockwise rotation of the rotatable knob 324 moves the threaded member 322 proximally and counterclockwise rotation of the rotatable knob 324 moves the threaded member 322 distally. The orientation of the internal and external threads on the rotatable knob 324 and the threaded member 322, respectively, determine which direction of rotation is tied to which direction of axial translation. Some suitable but non-limiting materials for the axial translation mechanism 320, the threaded member 322, and/or the rotatable knob 324 are described below.

In some embodiments, a distal pull ring (not shown) may be disposed within the distal portion 344 of the access sheath 340. In some embodiments, the distal pull ring may be disposed proximal to the second curve 348 and/or the distal tip 342. In some embodiments, the distal pull ring may be disposed proximate a distal end of the first curve 346. In some embodiments, the distal pull ring may be embedded within the access sheath 340. In some embodiments, the distal pull ring may be secured, bonded, and/or fixedly attached to an inner surface of the access sheath 340. Other configurations are also contemplated.

In some embodiments, the distal end of the access sheath 340 may be steerable using the rotatable knob 324 on the handle 310. A first steering wire 330 may extend through the access sheath 340 from the handle 310 and/or the handle housing 312 to the distal pull ring disposed adjacent the distal tip 342. A second steering wire 332 may extend through the access sheath 340 from the handle 310 and/or the handle housing 312 to the distal pull ring. The second steering wire 332 may be disposed on an opposite side of the access sheath 340 from the first steering wire 330 relative to a central longitudinal axis of the access sheath 340. Tension may be applied to the first steering wire 330 and/or the second steering wire 332 as described herein to bend and/or deflect the distal portion 344 and/or the first curve 346 of the access sheath 340 (e.g., FIG. 3). The first steering wire 330 may be configured to engage the axial translation mechanism 320 and/or the threaded member 322 to bend and/or deflect the distal portion 344 and/or the first curve 346 of the access sheath 340 in the first direction toward the handle 310 and/or the handle housing 312, toward and/or to the deflected configuration (e.g., FIG. 3). The second steering wire 332 may be configured to engage the axial translation mechanism 320 and/or the threaded member 322 to bend and/or deflect the distal portion 344 and/or the first curve 346 of the access sheath 340 in the second direction opposite the first direction and away from the handle 310 and/or the handle housing 312, toward and/or to the straightened configuration (e.g., FIG. 3).

In some embodiments, the access device 300 may include a pulley wheel 360 disposed within the handle 310 and/or the handle housing 312. The pulley wheel 360 may be engaged with the first steering wire 330 via a circumferential channel extending around the pulley wheel 360. In some embodiments, the access device 300 may include a tensioning member 370. The tensioning member 370 may couple a first end (e.g., a proximal end) of the first steering wire 330 to the handle 310 and/or to the handle housing 312. In some embodiments, the proximal end of the first steering wire 330 may be fixedly coupled to the handle 310 and/or the handle housing 312 by the tensioning member 370. In some embodiments, the pulley wheel 360 may engage the first steering wire 330 at a position proximal of the tensioning member 370. In some embodiments, the tensioning member 370 may be coupled to the handle 310 and/or the handle housing 312 at a position distal of the proximal end of the first steering wire 330. In some embodiments, the tensioning member 370 may be an elastic polymer, as shown in FIG. 4. In another example, the tensioning member 370 may be a coil spring. Other configurations are also contemplated.

The tensioning member 370 may be configured to apply a small, non-biasing amount of tension to the first steering wire 330 when the distal portion 344 and/or the first curve 346 of the access sheath 340 is disposed in the normal or relaxed configuration and/or when the distal portion 344 and/or the first curve 346 of the access sheath 340 is bent and/or deflected in the second direction, toward and/or to the straightened configuration. The purpose of the tensioning member 370 is to prevent the first steering wire 330 from disengaging from the pulley wheel 360 when there is no tension being applied to the first steering wire 330 by the axial translation mechanism 320 and/or the threaded member 322 (e.g., in the normal or relaxed configuration, or toward and/or in the straightened configuration) by holding the first steering wire 330 taught around the pulley wheel 360. Some suitable but non-limiting materials for the pulley wheel 360 and/or the tensioning member 370 are described below.

In addition or alternatively, in some embodiments, the access device 300 may include one or more ribs, projections, bosses, or posts extending transversely within the handle housing 312 between opposing walls and/or opposite sides of the handle housing 312. In some embodiments, the one or more ribs, projections, bosses, or posts may be disposed within the handle housing 312 at positions configured to approximate the diameter and/or the perimeter of the pulley wheel 360. In some embodiments, the one or more ribs, projections, bosses, or posts may replace the pulley wheel 360. In some embodiments, the one or more ribs, projections, bosses, or posts may be provided in addition to the pulley wheel 360. In some embodiments, the one or more ribs, projections, bosses, or posts may extend completely across an interior of the handle housing 312 from one side of the handle housing 312 to an opposing side of the handle housing 312. In some embodiments, the first steering wire 330 may be routed around and/or may slide past the one or more ribs, projections, bosses, or posts in a manner similar to the first steering wire 330 extending around the pulley wheel 360, such that the one or more ribs, projections, bosses, or posts may serve as guides for the first steering wire 330 and prevent loss of motion.

The threaded member 322 may include a first catch 326 extending transversely from the threaded member 322 in a first lateral direction. The first steering wire 330 may extend and/or pass through the first catch 326. The first steering wire 330 may include a first stop element 334 configured to engage with the axial translation mechanism 320 and/or the first catch 326 of the threaded member 322 when the threaded member 322 slides in a distal direction within the handle 310 and/or the handle housing 312 to apply tension to the first steering wire 330. The tension applied by the axial translation mechanism 320 and/or the threaded member 322 may be sufficient to overcome the self-bias of the access sheath 340 toward the normal or relaxed configuration and bend and/or deflect the distal portion 344 and/or the first curve 346 of the access sheath 340 in the first direction.

The threaded member 322 may include a second catch 328 extending transversely from the threaded member 322 in a second lateral direction opposite the first lateral direction. The second steering wire 332 may extend and/or pass through the second catch 328. The second steering wire 332 may include a second stop element 336 configured to engage with the axial translation mechanism 320 and/or the second catch 328 of the threaded member 322 when the threaded member 322 slides in a proximal direction within the handle 310 and/or the handle housing 312 to apply tension to the second steering wire 332. The tension applied by the axial translation mechanism 320 and/or the threaded member 322 may be sufficient to overcome the self-bias of the access sheath 340 toward the normal or relaxed configuration and bend and/or deflect the distal portion 344 and/or the first curve 346 of the access sheath 340 in the second direction.

The pulley wheel 360 permits the threaded member 322 to apply tension to both the first steering wire 330 and the second steering wire 332, depending upon which direction the threaded member 322 is moving. Tension applied to the first steering wire 330 and the second steering wire 332 causes bending and/or deflection of the distal portion 344 and/or the first curve 346 of the access sheath 340 away from the normal or relaxed configuration. Since both steering wires extend proximally from the distal pull ring, the pulley wheel 360 is needed to reverse the direction of the first steering wire 330 relative to the second steering wire 332 within the handle 310 and/or the handle housing 312 such that the threaded member 322 is able to selectively apply tension to both the first steering wire 330 and the second steering wire 332 by moving in opposite directions. In one or more alternative configurations, the handle 310 and/or the handle housing 312 may include an internal rib, an internal protrusion, or other features disposed therein, in place of the pulley wheel 360, around which the first steering wire 330 may extend and reverse direction to function as described herein.

When the threaded member 322 is disposed in a central position, the distal portion 344 and/or the first curve 346 of the access sheath 340 may be disposed in the normal or relaxed configuration. When the threaded member 322 is disposed in the central position, substantially no tension is being applied to the first steering wire 330 and/or the second steering wire 332. As the threaded member 322 is axially translated proximally and/or distally within the handle 310 and/or the handle housing 312, the threaded member 322 of the axial translation mechanism 320 may engage with the first steering wire 330 and/or the second steering wire 332 to apply tension thereto to bend and/or deflect the distal portion 344 and/or the first curve 346 of the access sheath 340 as described herein. Additionally, when the threaded member 322 is disposed in the central position, the first catch 326 may be engaged with the first stop element 334 but tension is not being applied to the first steering wire 330, and the second catch 328 may be engaged with the second stop element 336 but tension is not being applied to the second steering wire 332. As such, the central position of the threaded member 322 may be tension-neutral with respect to the first steering wire 330 and the second steering wire 332.

When the threaded member 322 is moved from the central position toward and/or until disposed in a proximal position, tension may be applied to the second steering wire 332 and the distal portion 344 and/or the first curve 346 of the access sheath 340 may be bent and/or deflected in the second direction away from the handle 310 and/or the handle housing 312, or toward and/or to the straightened configuration. In moving the threaded member 322 proximally within the handle 310 and/or the handle housing 312 from the central position, the second catch 328 engages the second stop element 336 and thereafter translates the second stop element 336 proximally, thereby applying tension to the second steering wire 332. The first stop element 334 may disengage from the axial translation mechanism 320, the threaded member 322, and/or the first catch 326 to release tension on the first steering wire 330 when the threaded member 322 slides in the proximal direction within the handle 310 and/or the handle housing 312. Accordingly, when the threaded member 322 is moved proximally from the central position, the first catch 326 may be disengaged from the first stop element 334 and the first catch 326 may slide proximally along and/or over the first steering wire 330. The first stop element 334 may be configured to float relative to (e.g., the first stop element 334 may not be directly fixed to) the axial translation mechanism 320, the threaded member 322, and/or the first catch 326 when the threaded member 322 slides in the proximal direction within the handle 310 and/or the handle housing 312. As such, slack would form in the first steering wire 330, which would allow the first steering wire 330 to disengage from the pulley wheel 360, except for the tension applied by the tensioning member 370. The tensioning member 370 holds the first steering wire 330 taught around the pulley wheel 360 while no tension is being applied to the first steering wire 330 by the threaded member 322 and/or the first catch 326. The tensioning member 370 merely absorbs any slack that would be formed in the first steering wire 330 due to the first catch 326 being disengaged from the first stop element 334 and prevents the first steering wire 330 from disengaging from the pulley wheel 360.

When the threaded member 322 is moved from the central position toward and/or until disposed in a distal position, tension may be applied to the first steering wire 330 and the distal portion 344 and/or the first curve 346 of the access sheath 340 may be bent and/or deflected in the first direction toward the handle 310 and/or the handle housing 312, or toward and/or to the deflected configuration. In moving the threaded member 322 distally within the handle 310 and/or the handle housing 312 from the central position, the first catch 326 engages the first stop element 334 and thereafter translates the first stop element 334 distally, thereby applying tension to the first steering wire 330. The second stop element 336 may disengage from the axial translation mechanism 320, the threaded member 322, and/or the second catch 328 to release tension on the second steering wire 332 when the threaded member 322 slides in the distal direction within the handle 310 and/or the handle housing 312. Accordingly, when the threaded member 322 is moved distally from the central position, the second catch 328 may be disengaged from the second stop element 336 and the second catch 328 may slide distally along and/or over the second steering wire 332. The second stop element 336 may be configured to float relative to (e.g., the second stop element 336 may not be directly fixed to) the axial translation mechanism 320, the threaded member 322, and/or the second catch 328 when the threaded member 322 slides in the distal direction within the handle 310 and/or the handle housing 312. As such, slack forms in the second steering wire 332, due to the second catch 328 being disengaged from the second stop element 336. As the threaded member 322 is translated distally from the proximal position and/or the central position, the first catch 326 engages the first stop element 334 and the first steering wire 330 is thereafter pulled around the pulley wheel 360 and tension applied by the tensioning member 370 is relieved as tension is instead applied to the first steering wire 330 by the first catch 326 and/or the threaded member 322.

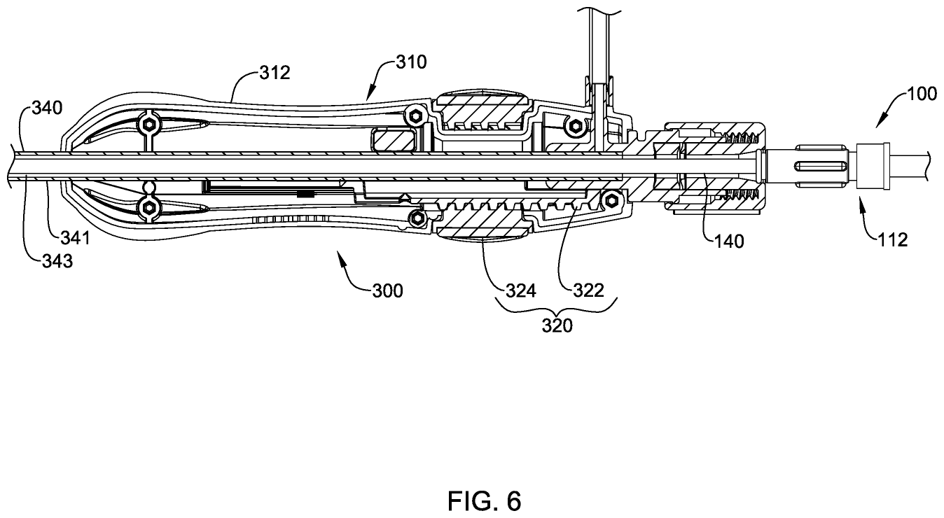

In some embodiments, the access sheath 340 may include a wall 341 (e.g., FIG. 6) defining a working lumen 343 (e.g., FIG. 6) extending longitudinally through the access sheath 340 and the handle 310. In some embodiments, the working lumen 343 may extend from a proximal end of the handle 310 to the distal tip 342 along the central longitudinal axis of the access sheath 340. In some embodiments, the working lumen 343 may be coaxial with the central longitudinal axis of the access sheath 340. In some embodiments, the working lumen 343 may be a guidewire lumen. In some embodiments, the working lumen 343 may be used to deliver a medical device or an occlusive implant. In some embodiments, the working lumen 343 may have multiple uses.

The access sheath 340 may include a plurality of steering wire lumens extending and/or disposed within the wall 341. In some embodiments, the plurality of steering wire lumens may include a first steering wire lumen and a second steering wire lumen. In some embodiments, the plurality of steering wire lumens may include more than two steering wire lumens. In some embodiments, the plurality of steering wire lumens may be oriented substantially parallel to the working lumen 343 and/or the central longitudinal axis of the access sheath 340. In some embodiments, the plurality of steering wire lumens may be disposed opposite each other and/or on opposite sides of the access sheath 340 relative to the working lumen 343 and/or the central longitudinal axis of the access sheath 340. Other configurations are also contemplated.

The first steering wire 330 and the second steering wire 332 may each be slidably disposed within the plurality of steering wire lumens. In one example, the first steering wire 330 may be slidably disposed within the first steering wire lumen and the second steering wire 332 may be disposed within the second steering wire lumen. The first steering wire 330 and the second steering wire 332 may be fixedly attached (e.g., bonded, welded, etc.) to the distal pull ring. For example, a distal end of the first steering wire 330 may be fixedly attached to the distal pull ring and a distal end of the second steering wire 332 may be fixedly attached to the distal pull ring at a position opposite the distal end of the first steering wire 330 relative to the central longitudinal axis of the access sheath 340. Some suitable but non-limiting materials for the first steering wire 330 and the second steering wire 332 are described below.

In some embodiments, clockwise rotation of the rotatable knob 324, as viewed proximally to distally, moves the threaded member 322 distally within the handle 310 and/or the handle housing 312, thereby applying tension to the first steering wire 330 and bending or deflecting the distal portion 344 and/or the first curve 346 of the access sheath 340 toward the handle 310 and/or the handle housing 312, or toward and/or to the deflected configuration. Counterclockwise rotation of the rotatable knob 324, as viewed proximally to distally, has moved the threaded member 322 proximally within the handle 310 and/or the handle housing 312, thereby applying tension to the second steering wire 332 and bending or deflecting the distal portion 344 and/or the first curve 346 of the access sheath 340 away from the handle 310 and/or the handle housing 312, or toward and/or to the straightened configuration. Other configurations are also contemplated.

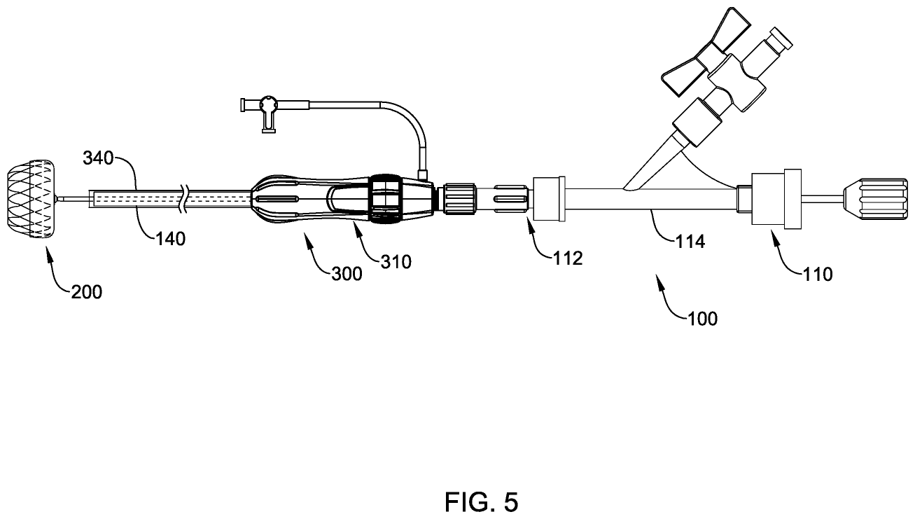

FIGS. 5-6 illustrate selected aspects of using the occlusive implant device 100 of FIGS. 1-2 with the access device 300 of FIGS. 3-4. To improve clarity and for the purpose of illustration only, the access device 300 is shown completely straight. However, it shall be understood that the access sheath 340 of the access device 300 may have a preset double curve, in the normal or relaxed configuration, as discussed herein. In some embodiments, as or when the occlusive implant 200 is deployed from the delivery sheath 140, the access sheath 340 of the access device 300 may be disposed in the normal or relaxed configuration, in the deflected configuration, or the straightened configuration, as described herein.

The access sheath 340 may be navigated through a patient's vasculature to a treatment site. For example, the access sheath 340 may be advanced to the patient's left atrium and the distal tip 342 disposed adjacent to the left atrial appendage. As may be seen in FIG. 5, the delivery sheath 140 of the occlusive implant device 100 may be disposed within the access sheath 340 of the access device 300. The distal end of the delivery sheath 140 may be disposed adjacent to and/or at the distal end of the access sheath 340. As such, the length of the delivery sheath 140 may be substantially equal to the length of the access device 300 (e.g., the combined length of the access sheath 340 and the handle 310). During use, the delivery sheath 140 may be advanced within the access sheath 340 with the occlusive implant 200 disposed therein in the delivery configuration. After the distal end of the delivery sheath 140 is disposed adjacent to and/or at the distal end of the access sheath 340, the core wire 130 may be advanced distally relative to the delivery sheath 140 to advance the occlusive implant 200 out of the delivery sheath 140 and the access sheath 340 at the treatment site, where the occlusive implant 200 may shift to the deployed configuration, as shown in FIG. 5.

FIG. 6 illustrates selected aspects of the access device 300 with the delivery sheath 140 of the occlusive implant device 100 extending through the access sheath 340. As may be seen in FIG. 6, the access sheath 340 may have a wall 341 defining the working lumen 343. The working lumen 343 may have a generally constant inner diameter along its length within the handle 310. This presents a smooth bore for transit of the delivery sheath 140 through the access sheath 340. The working lumen 343 must be sized to accommodate the delivery sheath 140 along its entire length. The access sheath 340 may have a generally constant outer diameter along its length, as seen in FIG. 5.

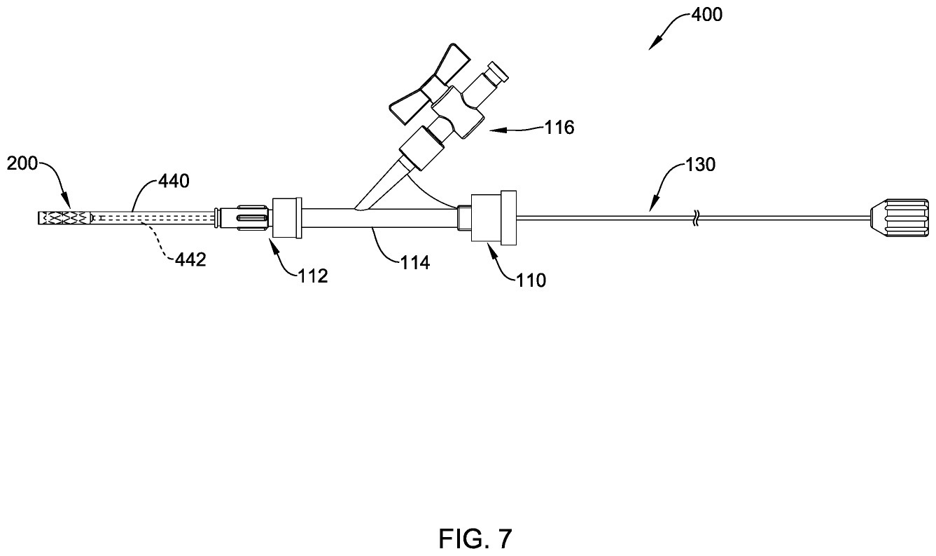

FIG. 7 illustrates selected aspects of an occlusive implant device 400 according to the current disclosure. The occlusive implant device 400 may be configured similarly to the occlusive implant device 100, and like elements are referenced using the same reference numerals.

The occlusive implant device 400 may include a delivery system including a delivery sheath 440 having a delivery lumen 442 extending proximally from a distal end of the delivery sheath 440. In one example, the delivery lumen 442 extends from a proximal opening to a distal opening of the delivery sheath 440. The delivery system may include a proximal hub 110. The delivery system may include a mid-hub 112. The delivery system may include a mid-shaft 114 extending from the proximal hub 110 to the mid-hub 112. The delivery sheath 440 may extend distally from the mid-hub 112. The delivery system may include a side port 116. The delivery system and/or the delivery lumen 442 may include a proximal segment (not shown) extending within and/or through the mid-hub 112, the mid-shaft 114, and the proximal hub 110. The proximal segment may be in fluid communication with and/or may be an extension of the delivery lumen 442 of the delivery sheath 440. The side port 116 may be in fluid communication with the proximal segment and/or the delivery lumen 142.

The occlusive implant device 400 may include a core wire 130 slidably and/or rotatably disposed within the delivery lumen 442 and the proximal segment. The occlusive implant device 400 may include the occlusive implant 200, as described herein, releasably attached to a distal end of the core wire 130. In some embodiments, a proximal end of the core wire 130 may extend proximally of a proximal end of the delivery sheath 440 and/or the proximal opening of the delivery lumen 442 for manual manipulation by a clinician or practitioner. Some suitable, but non-limiting, examples of materials for the occlusive implant device 400 and/or the delivery sheath 440, etc., including but not limited to metallic materials, polymeric materials, etc., are discussed below.

When compared to the delivery sheath 140 and the delivery lumen 142, the delivery sheath 440 and the delivery lumen 442 may be substantially shorter in length. For example, the delivery sheath 140 may have a length of about 32 inches from the mid-hub 112 to the distal end of the delivery sheath 140, whereas the delivery sheath 440 may have a length of about 10 inches, about 8 inches, about 6 inches, etc. from the mid-hub 112 to the distal end of the delivery sheath 440. Other configurations are also contemplated. In at least some embodiments, other elements and/or components of the occlusive implant device 100 and/or the delivery system thereof may remain substantially the same in the occlusive implant device 400 and/or the delivery system thereof. For example, the core wire 130 may remain unchanged, including its length.

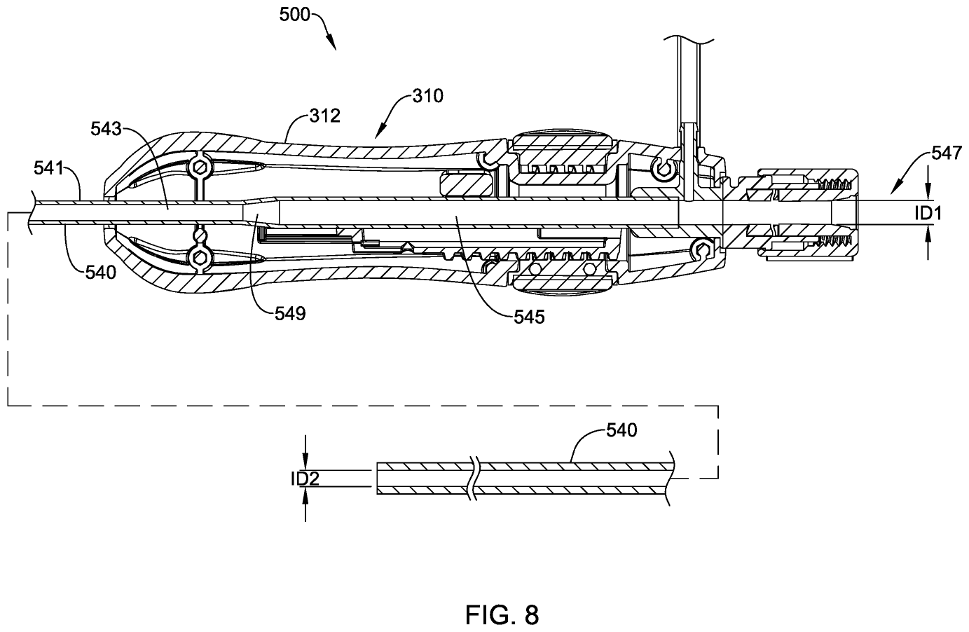

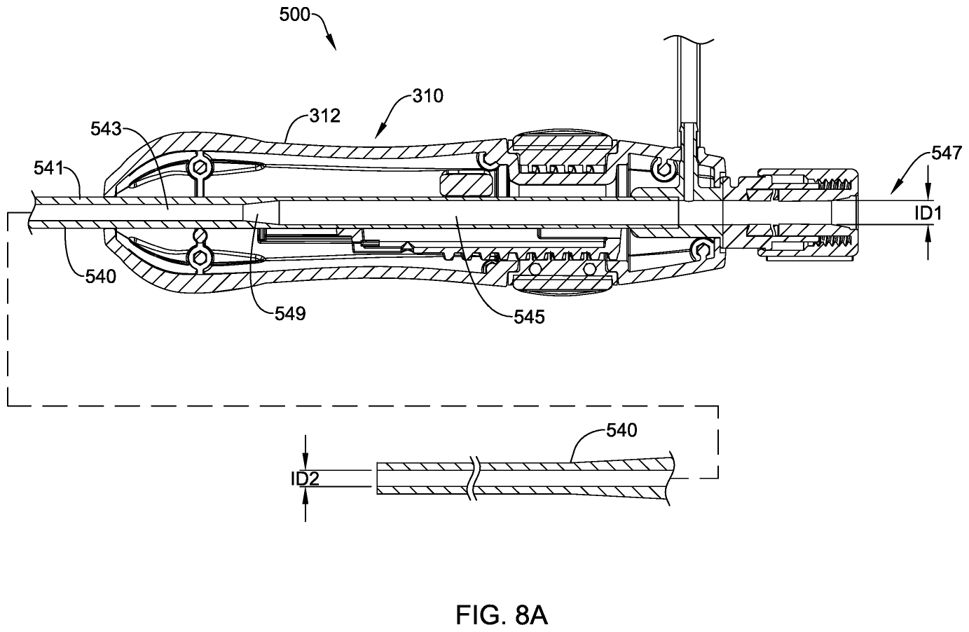

FIG. 8 illustrates selected aspects of an access device 500 according to the current disclosure. The access device 500 may be configured similarly to the access device 300, and like elements are referenced using the same reference numerals. Please refer to the discussion related to FIGS. 3-4 and the access device 300 for elements and/or components that are not expressly discussed herein and/or illustrated in FIG. 8. In one example, the access device 500 may be a bi-directional steerable catheter and/or an intravascular catheter. Examples of intravascular catheters may include, but are not limited to, balloon catheters, atherectomy catheters, device delivery catheters, drug delivery catheters, diagnostic catheters, and guide catheters. In some examples, the access device 500 may be a steerable access device having features, functions, and/or capabilities as described herein.

The access device 500 may include a handle 310 and an access sheath 540 extending distally from the handle 310. The access sheath 540 may be a generally tubular elongate member. In some embodiments, the access device 500 and/or the handle 310 may include at least one port 314 connected thereto. In some embodiments, the at least one port 314 may include a guidewire port, a side port, a fluid flush port, an imaging access port, or other suitable ports, access points, or functional features. The handle 310 may include a handle housing 312. The access sheath 540 may extend into and/or through a distal opening in the handle housing 312. In at least some embodiments, a proximal end of the access sheath 540 may be fixedly attached to and/or inside of the handle housing 312. In some embodiments, the access sheath 540 may have a normal or relaxed configuration. The access sheath 540 may be self-biased toward, and/or in the absence of any outside forces may return to, the normal or relaxed configuration. Some suitable but non-limiting materials for the handle 310 and/or the handle housing 312 are described below.

In some embodiments, the access sheath 540 may include a soft and/or atraumatic distal tip 342 as described herein. In some embodiments, the access sheath 540 may include a distal portion 344 having a first curve 346 and a second curve 348, such that the access sheath 540 has a preset double curve, in the normal or relaxed configuration. In some embodiments, the first curve 346 may be preset to curve upwards, as viewed from the side. Other configurations are also contemplated. In some embodiments, the second curve 348 may be preset to curve to the left, as viewed proximally to distally along the access sheath 540. Other configurations are also contemplated. In some embodiments, the distal portion 344 and/or the first curve 346 may be configured to bend or deflect in a first direction, wherein the distal tip 342 is bent and/or moved towards and/or closer to the handle 310, toward and/or to a deflected configuration, as shown in FIG. 3. In some embodiments, the distal portion 344 and/or the first curve 346 may be configured to bend or deflect in a second direction opposite the first direction, wherein the distal tip 342 is bent and/or moved away from and/or farther from the handle 310, toward and/or to a straightened configuration, as shown in FIG. 3. In some embodiments, the access sheath 540 may have only a single curve in the normal or relaxed configuration. In some embodiments, the access sheath 540 may be substantially straight in the normal or relaxed configuration.

In some embodiments, the access sheath 540 may be sized in accordance with its intended use. For example, the access sheath 540 can have a length that is in the range of about 10 to about 150 centimeters, about 25 to about 125 centimeters, about 50 to about 100 centimeters, about 25 centimeters to about 50 centimeters, about 50 to about 75 centimeters, about 75 to about 100 centimeters, etc. Other lengths are also contemplated, including but not limited to subsets of ranges disclosed herein. It is further contemplated that the outer diameter of the access sheath 540 distal of the handle 310 may vary based on the use or application. In some examples, the outer diameter of the access sheath 540 distal of the handle 310 may be about 2 millimeters (mm), about 3 mm (or 9 French), about 3.5 mm, about 4 mm (or 12 French), about 4.5 mm, about 5 mm (or 15 French), about 5.33 mm, about 5.5 mm, about 5.66 mm (or 17 French), about 6 mm, about 6.5 mm, about 7 mm (or 21 French), about 8 mm, or other suitable sizes. In some embodiments, the outer diameter of the access sheath 540 may be a maximum of 5.66 mm (17 French), and is preferably smaller than 5.66 mm (17 French). Other configurations are also contemplated. In some embodiments, it is desirable for the outer diameter of the access sheath 540 to be as small as possible. Some suitable but non-limiting materials for the access sheath 540 are described below.

The handle 310 may include an axial translation mechanism 320. The axial translation mechanism 320 may include a threaded member 322 slidably disposed within the handle 310 and/or the handle housing 312. The axial translation mechanism 320 may include a rotatable knob 324. In some embodiments, the rotatable knob 324 may be disposed about and/or may be configured to rotate about, and/or relative to, at least a portion of the handle 310 and/or the handle housing 312. The rotatable knob 324 may be configured to engage the threaded member 322 such that rotation of the rotatable knob 324 relative to the handle 310 and/or the handle housing 312 causes axial translation of the threaded member 322 proximally and/or distally within the handle 310 and/or the handle housing 312. In some embodiments, rotation of the rotatable knob 324 in a clockwise direction, as viewed along the access device 500 proximally to distally, may cause axial translation of the threaded member 322 distally within the handle 310 and/or the handle housing 312. In some embodiments, rotation of the rotatable knob 324 in a counterclockwise direction, as viewed along the access device 500 proximally to distally, may cause axial translation of the threaded member 322 proximally within the handle 310 and/or the handle housing 312. In some embodiments, the reverse and/or opposite configuration may be used, wherein clockwise rotation of the rotatable knob 324 moves the threaded member 322 proximally and counterclockwise rotation of the rotatable knob 324 moves the threaded member 322 distally. The orientation of the internal and external threads on the rotatable knob 324 and the threaded member 322, respectively, determine which direction of rotation is tied to which direction of axial translation. Some suitable but non-limiting materials for the axial translation mechanism 320, the threaded member 322, and/or the rotatable knob 324 are described below.

In some embodiments, a distal pull ring (not shown) may be disposed within the distal portion 344 of the access sheath 540. In some embodiments, the distal pull ring may be disposed proximal to the second curve 348 and/or the distal tip 342. In some embodiments, the distal pull ring may be disposed proximate a distal end of the first curve 346. In some embodiments, the distal pull ring may be embedded within the access sheath 540. In some embodiments, the distal pull ring may be secured, bonded, and/or fixedly attached to an inner surface of the access sheath 540. Other configurations are also contemplated.

In some embodiments, the distal end of the access sheath 540 may be steerable using the rotatable knob 324 on the handle 310. A first steering wire 330 may extend through the access sheath 540 from the handle 310 and/or the handle housing 312 to the distal pull ring disposed adjacent the distal tip 342. A second steering wire 332 may extend through the access sheath 540 from the handle 310 and/or the handle housing 312 to the distal pull ring. The second steering wire 332 may be disposed on an opposite side of the access sheath 540 from the first steering wire 330 relative to a central longitudinal axis of the access sheath 540. Tension may be applied to the first steering wire 330 and/or the second steering wire 332 as described herein to bend and/or deflect the distal portion 344 and/or the first curve 346 of the access sheath 540 (e.g., FIG. 3). The first steering wire 330 may be configured to engage the axial translation mechanism 320 and/or the threaded member 322 to bend and/or deflect the distal portion 344 and/or the first curve 346 of the access sheath 540 in the first direction toward the handle 310 and/or the handle housing 312, toward and/or to the deflected configuration (e.g., FIG. 3). The second steering wire 332 may be configured to engage the axial translation mechanism 320 and/or the threaded member 322 to bend and/or deflect the distal portion 344 and/or the first curve 346 of the access sheath 540 in the second direction opposite the first direction and away from the handle 310 and/or the handle housing 312, toward and/or to the straightened configuration (e.g., FIG. 3).

In some embodiments, the access device 500 may include a pulley wheel 360 disposed within the handle 310 and/or the handle housing 312. The pulley wheel 360 may be engaged with the first steering wire 330 via a circumferential channel extending around the pulley wheel 360. In some embodiments, the access device 500 may include a tensioning member 370. The tensioning member 370 may couple a first end (e.g., a proximal end) of the first steering wire 330 to the handle 310 and/or to the handle housing 312. In some embodiments, the proximal end of the first steering wire 330 may be fixedly coupled to the handle 310 and/or the handle housing 312 by the tensioning member 370. In some embodiments, the pulley wheel 360 may engage the first steering wire 330 at a position proximal of the tensioning member 370. In some embodiments, the tensioning member 370 may be coupled to the handle 310 and/or the handle housing 312 at a position distal of the proximal end of the first steering wire 330. In some embodiments, the tensioning member 370 may be an elastic polymer, as shown in FIG. 4. In another example, the tensioning member 370 may be a coil spring. Other configurations are also contemplated.

The tensioning member 370 may be configured to apply a small, non-biasing amount of tension to the first steering wire 330 when the distal portion 344 and/or the first curve 346 of the access sheath 540 is disposed in the normal or relaxed configuration and/or when the distal portion 344 and/or the first curve 346 of the access sheath 540 is bent and/or deflected in the second direction, toward and/or to the straightened configuration. The purpose of the tensioning member 370 is to prevent the first steering wire 330 from disengaging from the pulley wheel 360 when there is no tension being applied to the first steering wire 330 by the axial translation mechanism 320 and/or the threaded member 322 (e.g., in the normal or relaxed configuration, or toward and/or in the straightened configuration) by holding the first steering wire 330 taught around the pulley wheel 360. Some suitable but non-limiting materials for the pulley wheel 360 and/or the tensioning member 370 are described below.

In addition or alternatively, in some embodiments, the access device 500 may include one or more ribs, projections, bosses, or posts extending transversely within the handle housing 312 between opposing walls and/or opposite sides of the handle housing 312. In some embodiments, the one or more ribs, projections, bosses, or posts may be disposed within the handle housing 312 at positions configured to approximate the diameter and/or the perimeter of the pulley wheel 360. In some embodiments, the one or more ribs, projections, bosses, or posts may replace the pulley wheel 360. In some embodiments, the one or more ribs, projections, bosses, or posts may be provided in addition to the pulley wheel 360. In some embodiments, the one or more ribs, projections, bosses, or posts may extend completely across an interior of the handle housing 312 from one side of the handle housing 312 to an opposing side of the handle housing 312. In some embodiments, the first steering wire 330 may be routed around and/or may slide past the one or more ribs, projections, bosses, or posts in a manner similar to the first steering wire 330 extending around the pulley wheel 360, such that the one or more ribs, projections, bosses, or posts may serve as guides for the first steering wire 330 and prevent loss of motion.

The threaded member 322 may include a first catch 326 extending transversely from the threaded member 322 in a first lateral direction. The first steering wire 330 may extend and/or pass through the first catch 326. The first steering wire 330 may include a first stop element 334 configured to engage with the axial translation mechanism 320 and/or the first catch 326 of the threaded member 322 when the threaded member 322 slides in a distal direction within the handle 310 and/or the handle housing 312 to apply tension to the first steering wire 330. The tension applied by the axial translation mechanism 320 and/or the threaded member 322 may be sufficient to overcome the self-bias of the access sheath 540 toward the normal or relaxed configuration and bend and/or deflect the distal portion 344 and/or the first curve 346 of the access sheath 540 in the first direction.

The threaded member 322 may include a second catch 328 extending transversely from the threaded member 322 in a second lateral direction opposite the first lateral direction. The second steering wire 332 may extend and/or pass through the second catch 328. The second steering wire 332 may include a second stop element 336 configured to engage with the axial translation mechanism 320 and/or the second catch 328 of the threaded member 322 when the threaded member 322 slides in a proximal direction within the handle 310 and/or the handle housing 312 to apply tension to the second steering wire 332. The tension applied by the axial translation mechanism 320 and/or the threaded member 322 may be sufficient to overcome the self-bias of the access sheath 540 toward the normal or relaxed configuration and bend and/or deflect the distal portion 344 and/or the first curve 346 of the access sheath 540 in the second direction.

The pulley wheel 360 permits the threaded member 322 to apply tension to both the first steering wire 330 and the second steering wire 332, depending upon which direction the threaded member 322 is moving. Tension applied to the first steering wire 330 and the second steering wire 332 causes bending and/or deflection of the distal portion 344 and/or the first curve 346 of the access sheath 540 away from the normal or relaxed configuration. Since both steering wires extend proximally from the distal pull ring, the pulley wheel 360 is needed to reverse the direction of the first steering wire 330 relative to the second steering wire 332 within the handle 310 and/or the handle housing 312 such that the threaded member 322 is able to selectively apply tension to both the first steering wire 330 and the second steering wire 332 by moving in opposite directions. In one or more alternative configurations, the handle 310 and/or the handle housing 312 may include an internal rib, an internal protrusion, or other features disposed therein, in place of the pulley wheel 360, around which the first steering wire 330 may extend and reverse direction to function as described herein.

When the threaded member 322 is disposed in a central position, the distal portion 344 and/or the first curve 346 of the access sheath 540 may be disposed in the normal or relaxed configuration. When the threaded member 322 is disposed in the central position, substantially no tension is being applied to the first steering wire 330 and/or the second steering wire 332. As the threaded member 322 is axially translated proximally and/or distally within the handle 310 and/or the handle housing 312, the threaded member 322 of the axial translation mechanism 320 may engage with the first steering wire 330 and/or the second steering wire 332 to apply tension thereto to bend and/or deflect the distal portion 344 and/or the first curve 346 of the access sheath 540 as described herein. Additionally, when the threaded member 322 is disposed in the central position, the first catch 326 may be engaged with the first stop element 334 but tension is not being applied to the first steering wire 330, and the second catch 328 may be engaged with the second stop element 336 but tension is not being applied to the second steering wire 332. As such, the central position of the threaded member 322 may be tension-neutral with respect to the first steering wire 330 and the second steering wire 332.

When the threaded member 322 is moved from the central position toward and/or until disposed in a proximal position, tension may be applied to the second steering wire 332 and the distal portion 344 and/or the first curve 346 of the access sheath 540 may be bent and/or deflected in the second direction away from the handle 310 and/or the handle housing 312, or toward and/or to the straightened configuration. In moving the threaded member 322 proximally within the handle 310 and/or the handle housing 312 from the central position, the second catch 328 engages the second stop element 336 and thereafter translates the second stop element 336 proximally, thereby applying tension to the second steering wire 332. The first stop element 334 may disengage from the axial translation mechanism 320, the threaded member 322, and/or the first catch 326 to release tension on the first steering wire 330 when the threaded member 322 slides in the proximal direction within the handle 310 and/or the handle housing 312. Accordingly, when the threaded member 322 is moved proximally from the central position, the first catch 326 may be disengaged from the first stop element 334 and the first catch 326 may slide proximally along and/or over the first steering wire 330. The first stop element 334 may be configured to float relative to (e.g., the first stop element 334 may not be directly fixed to) the axial translation mechanism 320, the threaded member 322, and/or the first catch 326 when the threaded member 322 slides in the proximal direction within the handle 310 and/or the handle housing 312. As such, slack would form in the first steering wire 330, which would allow the first steering wire 330 to disengage from the pulley wheel 360, except for the tension applied by the tensioning member 370. The tensioning member 370 holds the first steering wire 330 taught around the pulley wheel 360 while no tension is being applied to the first steering wire 330 by the threaded member 322 and/or the first catch 326. The tensioning member 370 merely absorbs any slack that would be formed in the first steering wire 330 due to the first catch 326 being disengaged from the first stop element 334 and prevents the first steering wire 330 from disengaging from the pulley wheel 360.

When the threaded member 322 is moved from the central position toward and/or until disposed in a distal position, tension may be applied to the first steering wire 330 and the distal portion 344 and/or the first curve 346 of the access sheath 540 may be bent and/or deflected in the first direction toward the handle 310 and/or the handle housing 312, or toward and/or to the deflected configuration. In moving the threaded member 322 distally within the handle 310 and/or the handle housing 312 from the central position, the first catch 326 engages the first stop element 334 and thereafter translates the first stop element 334 distally, thereby applying tension to the first steering wire 330. The second stop element 336 may disengage from the axial translation mechanism 320, the threaded member 322, and/or the second catch 328 to release tension on the second steering wire 332 when the threaded member 322 slides in the distal direction within the handle 310 and/or the handle housing 312. Accordingly, when the threaded member 322 is moved distally from the central position, the second catch 328 may be disengaged from the second stop element 336 and the second catch 328 may slide distally along and/or over the second steering wire 332. The second stop element 336 may be configured to float relative to (e.g., the second stop element 336 may not be directly fixed to) the axial translation mechanism 320, the threaded member 322, and/or the second catch 328 when the threaded member 322 slides in the distal direction within the handle 310 and/or the handle housing 312. As such, slack forms in the second steering wire 332, due to the second catch 328 being disengaged from the second stop element 336. As the threaded member 322 is translated distally from the proximal position and/or the central position, the first catch 326 engages the first stop element 334 and the first steering wire 330 is thereafter pulled around the pulley wheel 360 and tension applied by the tensioning member 370 is relieved as tension is instead applied to the first steering wire 330 by the first catch 326 and/or the threaded member 322.

As seen in FIG. 8, in some embodiments, the access sheath 540 may include a wall 541 defining a working lumen 543 extending longitudinally through the access sheath 540 and the handle 310. In some embodiments, the working lumen 543 may extend from a proximal end of the handle 310 to the distal tip 342 along the central longitudinal axis of the access sheath 540. In some embodiments, the working lumen 543 may be coaxial with the central longitudinal axis of the access sheath 540. In some embodiments, the working lumen 543 may be a guidewire lumen. In some embodiments, the working lumen 543 may be used to deliver a medical device or an occlusive implant. In some embodiments, the working lumen 543 may have multiple uses.

A proximal portion of the working lumen 543 may define a garage section 545 connected to a proximal port 547 of the handle 310. A distal portion of the working lumen 543 may extend distally from the garage section 545 to a distal end of the access sheath 540 and/or to the distal tip 342 of the access sheath 540. The garage section 545 may have a first inner diameter ID1. The distal portion of the working lumen 543 may have a second inner diameter ID2 different from the first inner diameter ID1. In at least some embodiments, the second inner diameter ID2 may be less than the first inner diameter ID1.

In some embodiments, the working lumen 543 may have a tapered portion 549 extending between the distal portion of the working lumen 543 and the garage section 545. In some embodiments, the tapered portion 549 tapers from the first inner diameter ID1 proximate and/or at a proximal end of the tapered portion 549 to the second inner diameter ID2 proximate and/or at a distal end of the tapered portion 549. As such, the tapered portion 549 may taper radially inwardly in a distal direction. In some embodiments, the working lumen 543 tapers from the first inner diameter ID1 to the second inner diameter ID2 within the handle 310. As such, the garage section 545 may be disposed proximal of the distal end of the handle 310. The garage section 545 may be disposed and/or maintained entirely outside of the patient during a procedure.

In some embodiments, an outer surface and/or an outer diameter of the access sheath 540 and/or the wall 541 of the access sheath 540 may taper radially inward in a distal direction within the handle 310, as seen in FIG. 8. In some embodiments, the outer surface and/or the outer diameter of the access sheath 540 and/or the wall 541 of the access sheath 540 may taper radially inward in the distal direction at the same axial location as the tapered portion 549. In some embodiments, the outer surface of the access sheath 540 and/or the wall 541 of the access sheath 540 may be substantially parallel to an inner surface of the access sheath 540 and/or the wall 541 of the access sheath 540 along the length of the access sheath 540. In some embodiments, the wall 541 of the access sheath 540 may have a generally constant thickness along at least a portion of its length. In some embodiments, the wall 541 of the access sheath 540 may have a generally constant thickness along its entire length. In some alternative embodiments, an outer surface of the access sheath 540 and/or the wall 541 of the access sheath 540 may taper radially inward in a distal direction between the handle 310 and the distal end of the access sheath 540 and/or the distal tip 342 of the access sheath 540, as seen in FIG. 8A. As such, the access sheath 540 may have a reduced outer diameter compared to the access sheath 340 of FIGS. 3-4, which may facilitate easier navigation through the patient's vasculature, a smaller access hole into the vasculature, improved and/or easier transeptal crossing, and/or reduced ambulatory time for the patient, while permitting delivery of the same size of occlusive implant 200 that was delivered with the access sheath 340.

In some embodiments, the access sheath 540 and/or the working lumen 543 may be formed by reflowing the access sheath 540 over a tapered mandrel. In some embodiments, the access sheath 540 and/or the working lumen 543 may be formed by flaring a proximal end of a sheath having the second inner diameter ID2 and then injection molding a new proximal portion having the first inner diameter ID1 onto the flared proximal end to form the garage section 545. Other methods of manufacture are also contemplated. In some embodiments, the access sheath 540 may include a braided reinforcing member embedded within the access sheath 540. In some embodiments, the braided reinforcing member may be and/or form an intermediate layer of the access sheath 540. In some embodiments, the access sheath 540 may include a lubricious inner layer disposed on an inner surface of the wall 541. In one example, the lubricious inner layer may be a polymer such as polytetrafluoroethylene (PTFE). Other configurations and/or materials are also contemplated. In some embodiments, the access sheath 540 and/or the working lumen 543 may include a low friction coating disposed on the inner surface of the wall 541.

The access sheath 540 may include a plurality of steering wire lumens extending and/or disposed within the wall 541. In some embodiments, the plurality of steering wire lumens may include a first steering wire lumen and a second steering wire lumen. In some embodiments, the plurality of steering wire lumens may include more than two steering wire lumens. In some embodiments, the plurality of steering wire lumens may be oriented substantially parallel to the working lumen 543 and/or the central longitudinal axis of the access sheath 540. In some embodiments, the plurality of steering wire lumens may be disposed opposite each other and/or on opposite sides of the access sheath 540 relative to the working lumen 543 and/or the central longitudinal axis of the access sheath 540. Other configurations are also contemplated.

The first steering wire 330 and the second steering wire 332 may each be slidably disposed within the plurality of steering wire lumens. In one example, the first steering wire 330 may be slidably disposed within the first steering wire lumen and the second steering wire 332 may be disposed within the second steering wire lumen. The first steering wire 330 and the second steering wire 332 may be fixedly attached (e.g., bonded, welded, etc.) to the distal pull ring. For example, a distal end of the first steering wire 330 may be fixedly attached to the distal pull ring and a distal end of the second steering wire 332 may be fixedly attached to the distal pull ring at a position opposite the distal end of the first steering wire 330 relative to the central longitudinal axis of the access sheath 540. Some suitable but non-limiting materials for the first steering wire 330 and the second steering wire 332 are described below.

In some embodiments, clockwise rotation of the rotatable knob 324, as viewed proximally to distally, moves the threaded member 322 distally within the handle 310 and/or the handle housing 312, thereby applying tension to the first steering wire 330 and bending or deflecting the distal portion 344 and/or the first curve 346 of the access sheath 340 toward the handle 310 and/or the handle housing 312, or toward and/or to the deflected configuration. Counterclockwise rotation of the rotatable knob 324, as viewed proximally to distally, has moved the threaded member 322 proximally within the handle 310 and/or the handle housing 312, thereby applying tension to the second steering wire 332 and bending or deflecting the distal portion 344 and/or the first curve 346 of the access sheath 540 away from the handle 310 and/or the handle housing 312, or toward and/or to the straightened configuration. Other configurations are also contemplated.

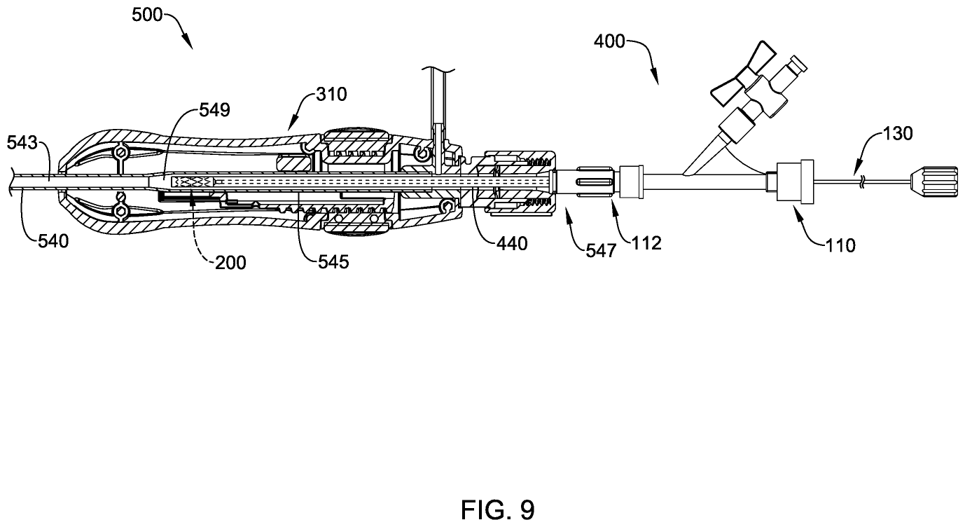

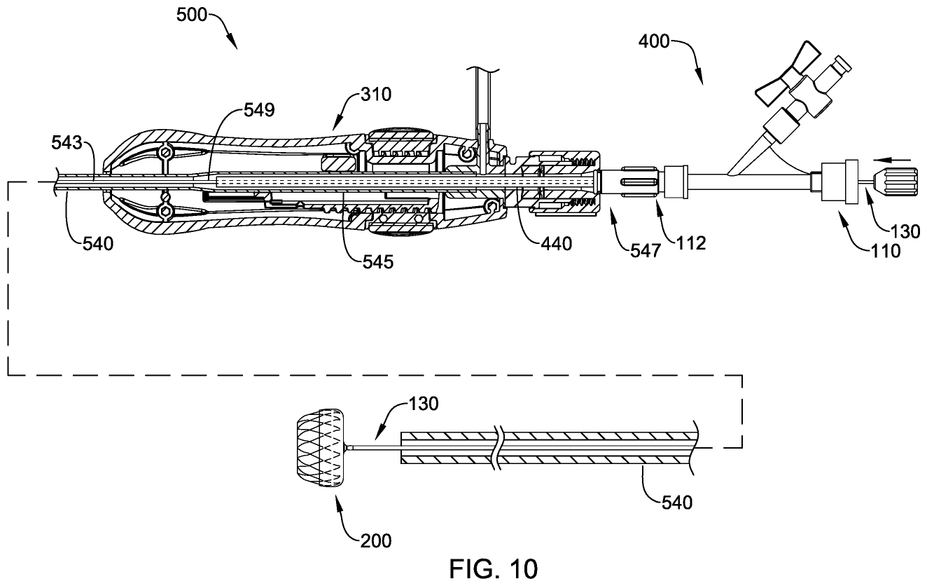

FIGS. 9-10 are partial cross-sectional views illustrating selected aspects of an occlusive implant system and a method of delivering an occlusive implant to a treatment site using the occlusive implant device 400 of FIG. 7 with the access device 500 of FIG. 8.

The method may include advancing the distal end of the access device 500 to the treatment site, as is known in the art. As discussed herein, the proximal portion of the working lumen 543 defines the garage section 545 connected to the proximal port 547 of the handle 310. The proximal portion of the working lumen 543 and/or the garage section 545 may have a first inner diameter ID1, as described herein.

The method may include inserting the delivery sheath 440 into the garage section 545 of the access device 500. The delivery sheath 440 may be configured to be slidably received within the garage section 545 of the access device 500. In some embodiments, the delivery sheath 440 may have an outer diameter that is less than the first inner diameter ID1. In some embodiments, the outer diameter of the delivery sheath 440 may be about 10% less, about 7.5% about 5% less, about 4% less, about 3% less, about 2% less, about 1% less, etc. than the first inner diameter ID1. In one example, the outer diameter of the delivery sheath 440 may be about 0.005 inches less than the first inner diameter ID1. Other configurations are also contemplated.

As may be seen in FIGS. 9-10, the distal end of the delivery sheath 440 may be disposed proximate the distal end of the garage section 545 and/or proximate the tapered portion 549 when the delivery sheath 440 is disposed within the garage section 545 and the mid-hub 112 is positioned directly adjacent to and/or within the proximal port 547 of the handle 310. The delivery sheath 440 may have a first length from a distal end of the mid-hub 112 to the distal end of the delivery sheath 440. The garage section 545 may have a second length from the proximal port 547 to the distal end of the garage section 545 and/or the tapered portion 549. In some embodiments, the second length may be less than 10% greater than the first length. In some embodiments, the second length may be less than 5% greater than the first length. In some embodiments, the second length may be substantially equal to the first length. Other configurations are also contemplated.

In some embodiments, the garage section 545 is less than 10 inches in length from the proximal port 547 to a distal end of the garage section 545. In some embodiments, the garage section 545 is less than 10 inches in length from the proximal port 547 to a proximal end of the tapered portion 549. In some embodiments, the garage section 545 is less than 8 inches in length from the proximal port 547 to a distal end of the garage section 545. In some embodiments, the garage section 545 is less than 8 inches in length from the proximal port 547 to a proximal end of the tapered portion 549. In some embodiments, the garage section 545 is about 6 inches in length from the proximal port 547 to a distal end of the garage section 545. In some embodiments, the garage section 545 is about 6 inches in length from the proximal port 547 to a proximal end of the tapered portion 549. Other configurations are also contemplated.

In at least some embodiments, the core wire 130 may have a length greater than a combined length of the delivery system (e.g., from the proximal hub 110 to the distal end of the delivery sheath 440) and the access sheath 540 when the distal end of the delivery sheath 440 is disposed within the garage section 545. In some embodiments, the core wire 130 may have a length greater than the combined length of the delivery system (e.g., from the proximal hub 110 to the distal end of the delivery sheath 440) and the access sheath 540 when the distal end of the delivery sheath 440 is disposed proximate the distal end of the garage section 545 and/or proximate the tapered portion 549. In some embodiments, the core wire 130 may have a length greater than the combined length of the delivery system (e.g., from the proximal hub 110 to the distal end of the delivery sheath 440) and the access sheath 540 when the delivery sheath 440 is disposed within the garage section 545 and the mid-hub 112 is positioned directly adjacent to and/or within the proximal port 547 of the handle 310.

As seen in FIG. 10, the method may include advancing the core wire 130 distally through the delivery lumen 442 of the delivery sheath 440 and into the working lumen 543 of the access sheath 540 to advance the occlusive implant 200 through the working lumen 543 and out the distal end of the access sheath 540 at the treatment site. In one example, the method may include advancing the core wire 130 distally through the delivery lumen 442 and into the working lumen 543 of the access sheath 540 to advance the occlusive implant 200 (e.g., a left atrial appendage closure device) through the working lumen 543 and out the distal end of the access sheath 540 at the treatment site (e.g., into the left atrial appendage). The occlusive implant 200 may be configured to shift to the deployed configuration when unconstrained by the delivery sheath 440 and/or the access sheath 540.

In at least some embodiments, the distal end of the delivery sheath 440 may be prevented from extending distal of the garage section 545 of the access device 500. In some embodiments, the second length of the garage section 545 is at least as long as the first length of the delivery sheath 440, as those lengths are described herein. In some embodiments, the mid-hub 112 may abut a proximal end and/or a proximal face of the proximal port 547 of the access device 500 and/or the handle 310, thus preventing the delivery system and/or the delivery sheath 440 from extending distal of the garage section 545. In some embodiments, the mid-hub 112 may be axially secured to the proximal port 547 of the access device 500 and/or the handle 310, such as with a connector or other means, thus preventing the delivery system and/or the delivery sheath 440 from moving relative to the access device 500 and similarly preventing the delivery system and/or the delivery sheath 440 from extending distal of the garage section 545.

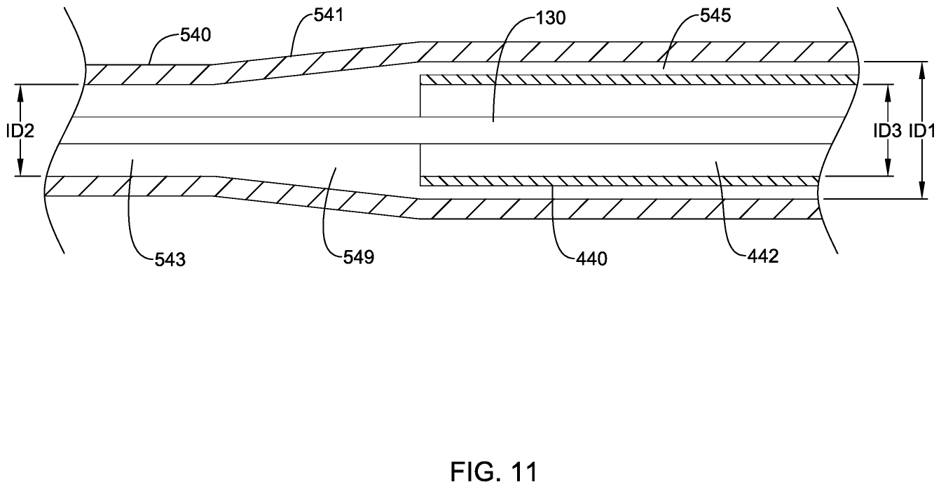

FIG. 11 illustrates selected aspects of the occlusive implant system of FIGS. 9-10 related to interaction between the occlusive implant device 400 and the access device 500. More specifically, FIG. 11 shows selected aspects related to the delivery sheath 440 of the occlusive implant device 400 positioned within the garage section 545 of the access device 500. In FIG. 11, the core wire 130 is shown extending through the delivery sheath 440, the access sheath 540, and/or the working lumen 543, such as after the core wire 130 and the occlusive implant 200 have been advanced out the distal end of the access sheath 540 (e.g., FIG. 10).

As discussed herein, the working lumen 543 may include the tapered portion 549 extending between the distal portion of the working lumen 543 and the garage section 545. The distal end of the delivery sheath 440 may be disposed and/or positioned proximate the distal end of the garage section 545 and/or the proximal end of the tapered portion 549. In some embodiments, the delivery sheath 440 may have an outer diameter less than the first inner diameter ID1 of the garage section 545 and greater than the second inner diameter ID2 of the distal portion of the working lumen 543. In some alternative configurations, the distal end of the delivery sheath 440 may be prevented from advancing distal of the garage section 545 by the tapered portion 549. In some embodiments, there may be a small clearance between the outer diameter of the delivery sheath 440 and the first inner diameter ID1 of the garage section 545 such that the delivery sheath 440 may be slidably received within the garage section 545, as discussed herein.

In some embodiments, the distal portion of the delivery lumen 442 and/or the delivery sheath 440 may have a third inner diameter ID3. In some embodiments, the third inner diameter ID3 may be substantially equal to the second inner diameter ID2 of the distal portion of the working lumen 543. In some embodiments, the third inner diameter ID3 may be less than the second inner diameter ID2 of the distal portion of the working lumen 543. In some embodiments, the third inner diameter ID3 may be less than 5% greater than the second inner diameter ID2 of the distal portion of the working lumen 543. In some embodiments, the third inner diameter ID3 may be substantially equal to or less than the second inner diameter ID2 of the distal portion of the working lumen 543.

Accordingly, in some embodiments, the delivery sheath 440 may have an outer diameter greater than the second inner diameter ID2 of the distal portion of the working lumen 543 and the delivery lumen 442 may have a third inner diameter ID3 substantially equal to or less than the second inner diameter ID2 of the distal portion of the working lumen 543. Other configurations are also contemplated.

In some embodiments, the tapered portion 549 may be sized and configured to guide the occlusive implant 200 from the distal portion of the delivery lumen 442 and/or the delivery sheath 440 into the distal portion of the working lumen 543 as the core wire 130 is advanced distally while minimizing friction and/or resistance between the occlusive implant 200 and the wall 541 of the working lumen 543.

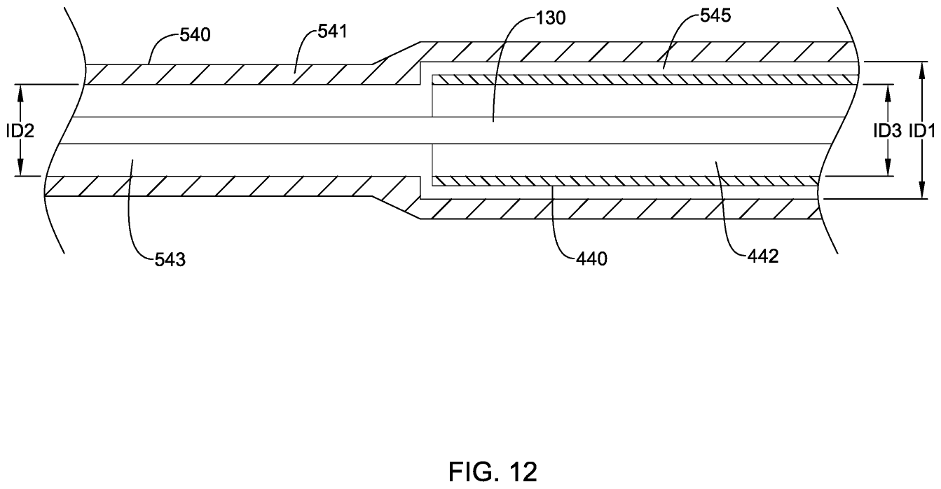

FIG. 12 illustrates selected aspects of an alternative configuration of the occlusive implant system of FIGS. 9-11 related to interaction between the occlusive implant device 400 and the access device 500. More specifically, FIG. 12 shows selected aspects related to the delivery sheath 440 of the occlusive implant device 400 positioned within the garage section 545 of the access device 500. In FIG. 12, the core wire 130 is shown extending through the delivery sheath 440, the access sheath 540, and/or the working lumen 543, such as after the core wire 130 and the occlusive implant 200 have been advanced out the distal end of the access sheath 540 (e.g., FIG. 10).

In the alternative configuration of FIG. 12, the working lumen 543 may include a step change in inner diameter between the distal portion of the working lumen 543 and the garage section 545. The distal end of the delivery sheath 440 may be disposed and/or positioned proximate the distal end of the garage section 545 and/or the proximal end of the step change in inner diameter from the first inner diameter ID1 to the second inner diameter ID2. In some embodiments, the delivery sheath 440 may have an outer diameter less than the first inner diameter ID1 of the garage section 545 and greater than the second inner diameter ID2 of the distal portion of the working lumen 543. In some alternative configurations, the distal end of the delivery sheath 440 may be prevented from advancing distal of the garage section 545 by the step change in inner diameter between the distal portion of the working lumen 543 and the garage section 545. In some embodiments, there may be a small clearance between the outer diameter of the delivery sheath 440 and the first inner diameter ID1 of the garage section 545 such that the delivery sheath 440 may be slidably received within the garage section 545, as discussed herein.

In some embodiments, the distal portion of the delivery lumen 442 and/or the delivery sheath 440 may have a third inner diameter ID3. In some embodiments, the third inner diameter ID3 may be substantially equal to the second inner diameter ID2 of the distal portion of the working lumen 543. In some embodiments, the third inner diameter ID3 may be less than the second inner diameter ID2 of the distal portion of the working lumen 543. In some embodiments, the third inner diameter ID3 may be substantially equal to or less than the second inner diameter ID2 of the distal portion of the working lumen 543.

Accordingly, in some embodiments, the delivery sheath 440 may have an outer diameter greater than the second inner diameter ID2 of the distal portion of the working lumen 543 and the delivery lumen 442 may have a third inner diameter ID3 substantially equal to or less than the second inner diameter ID2 of the distal portion of the working lumen 543. Other configurations are also contemplated.

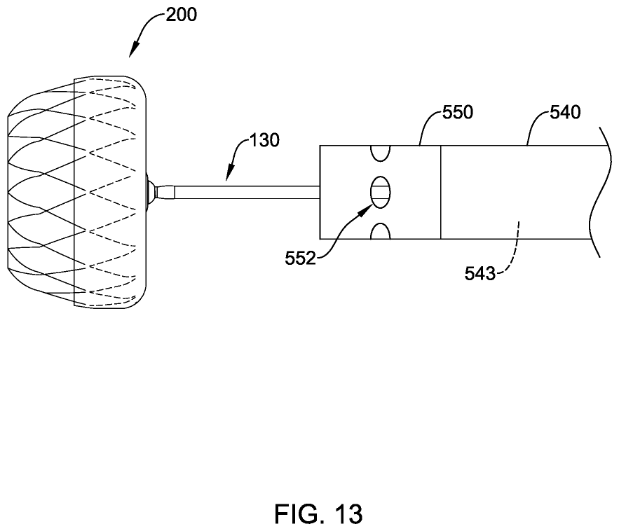

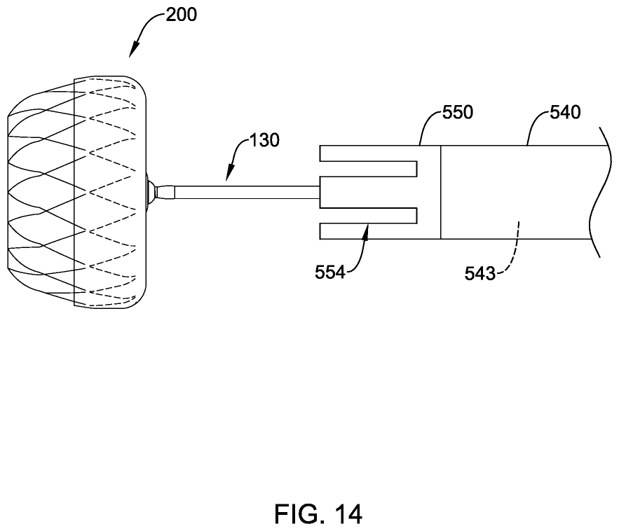

In some embodiments, the access sheath 540 may be configured to permit recapture of the occlusive implant 200 during a procedure. In some embodiments, the distal end of the access sheath 540 may include a soft distal tip 550, as seen in FIGS. 13-14. In some embodiments, the soft distal tip 550 may be formed from a material having a lower durometer rating than a remainder of the access sheath 540. In some embodiments, the soft distal tip 550 may be at least 10% softer (e.g., have a durometer rating at least 10% less) than the remainder of the access sheath 540. In some embodiments, the soft distal tip 550 may be at least 25% softer (e.g., have a durometer rating at least 25% less) than the remainder of the access sheath 540. In some embodiments, the soft distal tip 550 may be at least 50% softer (e.g., have a durometer rating at least 50% less) than the remainder of the access sheath 540. Other relative durometer ratings and/or differences are also contemplated. In some embodiments, the soft distal tip 550 may be devoid of the braided reinforcing member and/or the lubricious inner layer. Other configurations are also contemplated.

In some embodiments, the soft distal tip 550 may optionally include at least one aperture 552 extending radially through a wall of the soft distal tip 550, as seen in FIG. 13. In some embodiments, the at least one aperture 552 may be formed in one or more of a variety of shapes, including but not limited to, circles, ovals, squares, rectangles, polygons, etc., and may include combinations of shapes. The at least one aperture 552 may permit the soft distal tip 550 to resiliently flex and/or deform axially and/or radially to prevent damage to the occlusive implant 200 as the occlusive implant 200 is pulled proximally back into the working lumen 543 by the core wire 130. Other configurations, uses for, and/or benefits from the at least one aperture 552 are also contemplated.

In some embodiments, the soft distal tip 550 may optionally include at least one axial slit 554 extending through the wall of the soft distal tip 550, as seen in FIG. 14. The at least one axial slit 554 may extend proximally from the distalmost end of the soft distal tip 550. In some embodiments, the at least one axial slit 554 may be formed in one or more of a variety of widths and/or shapes such as straight, curvilinear, sinusoidal, etc. The at least one axial slit 554 may permit the soft distal tip 550 to resiliently flex and/or deform axially and/or radially to prevent damage to the occlusive implant 200 as the occlusive implant 200 is pulled proximally back into the working lumen 543 by the core wire 130. Other configurations, uses for, and/or benefits from the at least one axial slit 554 are also contemplated.

The materials that can be used for the various components of the occlusive implant system (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the access device, the access sheath, the occlusive implant device, the delivery sheath, the occlusive implant, the core wire, the expandable framework, the occlusive element, etc. and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable metals and metal alloys include stainless steel, such as 444V, 444L, and 314LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; combinations thereof; and the like; or any other suitable material.

In at least some embodiments, portions or all of the system and/or other elements disclosed herein may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids a user in determining the location and/or orientation of the system and/or other elements disclosed herein. Some examples of radiopaque materials can include, but are not limited to, gold, platinum, palladium, tantalum, tungsten alloy, polymer material loaded with a radiopaque filler, and the like.

In some embodiments, a degree of Magnetic Resonance Imaging (MM) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an Mill image. The system or portions thereof, may also be made from a material that the MM machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the system and/or other elements disclosed herein may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN®), polyether block ester, polyurethane (for example, Polyurethane 85A), polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps, without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An occlusive implant system, comprising:
an access device including a handle and an access sheath extending distally from the handle, wherein the access device includes a working lumen extending longitudinally through the access sheath and the handle;
wherein a proximal portion of the working lumen defines a garage section connected to a proximal port of the handle, and a distal portion of the working lumen extends distally of the garage section to a distal end of the access sheath; and
an occlusive implant device including:
a delivery system including a delivery sheath configured to be slidably received within the garage section, the delivery sheath having a delivery lumen extending proximally from a distal end of the delivery sheath;
a core wire slidably disposed within the delivery lumen; and
an occlusive implant releasably attached to a distal end of the core wire, the occlusive implant being configured to shift between a delivery configuration and a deployed configuration;
wherein the occlusive implant is disposed within a distal portion of the delivery lumen in the delivery configuration;
wherein the garage section has a first inner diameter and the distal portion of the working lumen has a second inner diameter less than the first inner diameter;
wherein the delivery sheath has an outer diameter less than the first inner diameter and greater than the second inner diameter.

2. The occlusive implant system of claim 1, wherein the distal portion of the delivery lumen has a third inner diameter and the third inner diameter is substantially equal to or less than the second inner diameter.

3. The occlusive implant system of claim 1, wherein the working lumen includes a tapered portion extending between the distal portion of the working lumen and the garage section.

4. The occlusive implant system of claim 3, wherein the tapered portion tapers from the first inner diameter proximate a proximal end of the tapered portion to the second inner diameter proximate a distal end of the tapered portion.

5. The occlusive implant system of claim 1, wherein the delivery system includes:
a proximal hub;
a mid-hub; and
a mid-shaft extending from the proximal hub to the mid-hub;
wherein the delivery sheath extends distally from the mid-hub.

6. The occlusive implant system of claim 5, wherein the delivery sheath has a first length from the mid-hub to the distal end of the delivery sheath;
wherein the garage section has a second length from the proximal port to a distal end of the garage section;
wherein the second length is less than 10% greater than the first length.

7. The occlusive implant system of claim 6, wherein the second length is less than 5% greater than the first length.

8. The occlusive implant system of claim 5, wherein the distal end of the delivery sheath is disposed proximate a distal end of the garage section when the delivery sheath is disposed within the garage section and the mid-hub is positioned adjacent the proximal port of the handle.

9. The occlusive implant system of claim 1, wherein the core wire has a length greater than a combined length of the delivery system and the access sheath when the distal end of the delivery sheath is disposed within the garage section.

10. An occlusive implant system, comprising:
a steerable access device including a handle and an access sheath extending distally from the handle, wherein the steerable access device includes a working lumen extending longitudinally through the access sheath and the handle;
wherein a proximal portion of the working lumen defines a garage section connected to a proximal port of the handle, and a distal portion of the working lumen extends distally of the garage section to a distal end of the access sheath; and
an occlusive implant device including:
a delivery system including a delivery sheath configured to be slidably received within the garage section, the delivery sheath having a delivery lumen extending proximally from a distal end of the delivery sheath;
a core wire slidably disposed within the delivery lumen; and
an occlusive implant releasably attached to a distal end of the core wire, the occlusive implant being configured to shift between a delivery configuration and a deployed configuration;
wherein the occlusive implant is disposed within a distal portion of the delivery lumen in the delivery configuration;
wherein the garage section has a first inner diameter and the distal portion of the working lumen has a second inner diameter different from the first inner diameter.

11. The occlusive implant system of claim 10, wherein the working lumen changes from the first inner diameter to the second inner diameter within the handle.

12. The occlusive implant system of claim 10, wherein the garage section is disposed proximal of a distal end of the handle.

13. The occlusive implant system of claim 10, wherein the garage section is less than 10 inches in length from the proximal port to a distal end of the garage section.

14. The occlusive implant system of claim 10, wherein the distal end of the delivery sheath is prevented from extending distal of the garage section.

15. A method of delivering an occlusive implant to a treatment site, comprising:
advancing a distal end of an access device to the treatment site, the access device including a handle, an access sheath extending distally from the handle, and a working lumen extending through the access sheath and the handle;
wherein a proximal portion of the working lumen defines a garage section connected to a proximal port of the handle and having a first inner diameter, and a distal portion of the working lumen extends distally of the garage section to a distal end of the access sheath and has a second inner diameter less than the first inner diameter;
inserting a delivery sheath into the garage section and positioning a distal end of the delivery sheath proximate a distal end of the garage section, the delivery sheath having a delivery lumen extending proximally from the distal end of the delivery sheath;
wherein an occlusive implant is disposed within a distal portion of the delivery lumen in a delivery configuration, and a core wire releasably attached to the occlusive implant is slidably disposed within the delivery lumen; and
advancing the core wire distally through the delivery lumen and into the working lumen to advance the occlusive implant through the working lumen and out the distal end of the access device at the treatment site;
wherein the occlusive implant is configured to shift to a deployed configuration when unconstrained.

16. The method of claim 15, wherein the working lumen tapers from the first inner diameter to the second inner diameter within the handle.

17. The method of claim 15, wherein an outer diameter of the access sheath tapers radially inward in a distal direction within the handle.

18. The method of claim 15, wherein the distal end of the access sheath is steerable using a knob on the handle.

19. The method of claim 15, wherein the occlusive implant is a left atrial appendage closure device.

20. The method of claim 15, wherein the delivery sheath has an outer diameter greater than the second inner diameter and the delivery lumen has a third inner diameter substantially equal to or less than the second inner diameter.
